(19) United States
(12) Patent Application Publication
(10) Pub. No.: US 2020/0407758 A1
(43) Pub. Date: Dec. 31, 2020

(54) ALPHA-AMYLASES FOR COMBINATION WITH GLUCOAMYLASES FOR IMPROVING SACCHARIFICATION

(71) Applicant: Lallemand Hungary Liquidity Management LLC, Budapest (HU)

(72) Inventors: Ryan Skinner, South Royalton, VT (US); Charles F. Rice, Hopkinton, NH (US); Aaron Argyros, Etna, NH (US)

(21) Appl. No.: 16/314,346

(22) PCT Filed: Jun. 30, 2017
(86) PCT No.: PCT/EP2017/066378

§ 371 Date: Dec. 28, 2018

Related U.S. Application Data
(60) Provisional application No. 62/357,664, filed on Jul. 1, 2016.

(51) Int. Cl. C12P 7/10; C12N 1/16; C12N 9/30; C12N 9/34

(52) U.S. Cl. CPC C12P 7/10; C12N 1/16; C12N 9/242; C12R 1/865; C12Y 302/01001; C12Y 302/01003; C12N 9/2428

(57) ABSTRACT

The present disclosure relates to alpha-amylases for use in combination with glucoamylases for improving the hydrolysis of a raw starch. The alpha-amylases can be provided in a purified form and/or can be expressed from a recombinant host cell. The present disclosure also provides a population of recombinant host cells expressing the alpha-amylases to be used in combination with recombinant host cells expressing the glucoamylases.

### CROSS-REFERENCE TO RELATED APPLICATIONS AND DOCUMENTS

The present application claims priority from U.S. provisional patent application 62/357,664 filed Jul. 1, 2016 and incorporated herewith in its entirety. An electronic sequence listing is concurrently filed herewith and the content of this sequence listing is incorporated by reference in the present application.

### TECHNICAL FIELD

The present disclosure relates to alpha-amylases that can be used, in combination with glucoamylases, for improving the hydrolysis of starch in a lignocellulosic material, such as corn.

### BACKGROUND

Saccharomyces cerevisiae is the primary biocatalyst used in the commercial production of fuel ethanol. This organism is proficient in fermenting glucose to ethanol, often to concentrations greater than 20% w/v. However, S. cerevisiae lacks the ability to hydrolyze polysaccharides and therefore requires the exogenous addition of purified enzymes to convert complex sugars to glucose. For example, in the United States, the primary source of fuel ethanol is corn starch, which, regardless of the mashing process, requires the exogenous addition of both alpha-amylase and glucoamylase. The cost of the purified enzymes range from $0.02-0.04 per gallon, which at 14 billion gallons of ethanol produced each year, represents a substantial cost savings opportunity for producers if they could reduce their enzyme dose.

In a broad sense, there are two major fermentation processes in the corn ethanol industry: liquefied corn mash and raw corn flour. In the mash process, corn is both thermally and enzymatically liquefied using alpha-amylases prior to fermentation in order to break down long chain starch polymers into smaller dextrins. The mash is then cooled and inoculated with S. cerevisiae along with the exogenous addition of purified glucoamylase, an exo-acting enzyme which will further break down the dextrin into utilizable glucose molecules. In the raw flour process, the corn is only milled, not heated, creating a raw flour-like substrate which relies heavily on the addition of exogenous enzymes to complete the saccharification process.

It would be desirable to be provided with improved alpha-amylases for the hydrolysis of raw starch. It would further be desirable to reduce the need for external enzyme addition during the saccharification process, particularly during ethanol fermentation.

### BRIEF SUMMARY

The present disclosure relates to the combination of alpha-amylases and glucoamylases for the hydrolysis of raw starch.

In a first aspect, the present disclosure concerns a first polypeptide having alpha-amylase activity for use in combination with a second polypeptide having glucoamylase activity on raw starch, wherein:
the first polypeptide is an alpha-amylase polypeptide, an alpha-amylase variant and/or an alpha-amylase fragment;
the alpha-amylase polypeptide has the amino acid sequence of SEQ ID NO: 2;
the alpha-amylase variant has at least 70% identity with the alpha-amylase polypeptide and has alpha-amylase activity;
the alpha-amylase fragment has at least 70% identity with the alpha-amylase polypeptide and has alpha-amylase activity; and
the first polypeptide is provided in a purified form or is expressed from a first recombinant host cell comprising a first genetic modification allowing the production of the alpha-amylase polypeptide, the alpha-amylase variant or the alpha-amylase fragment.

In an embodiment, the first polypeptide is expressed from the first recombinant host cell, such as, for example a recombinant yeast host cell (e.g., from the genus Saccharomyces or from the species Saccharomyces cerevisiae). In another embodiment, the polypeptide having glucoamylase activity is a glucoamylase variant and/or a glucoamylase fragment and wherein:
the glucoamylase polypeptide has the amino acid sequence of SEQ ID NO: 5 or 6;
the glucoamylase variant has at least 70% identity with the glucoamylase polypeptide and has glucoamylase activity; and
the glucoamylase fragment has at least 70% identity with the glucoamylase polypeptide and has glucoamylase activity.

In yet another embodiment, the first recombinant host cell further comprises a second genetic modification selected from the group consisting of a genetic modification for reducing the production of one or more native enzymes that function to produce glycerol or regulate glycerol synthesis, a genetic modification for allowing the production of the second polypeptide having glucoamylase activity and a genetic modification for reducing the production of one or more native enzymes that function to catabolize formate. For example, the first recombinant host cell can have the genetic modification for reducing the production of one or more native enzymes that function to produce glycerol or regulate glycerol synthesis. In yet another embodiment, the genetic modification for reducing the production of one or more native enzymes that function to produce glycerol or regulate glycerol synthesis is a reduction in the expression of the gene encoding the GPD2 polypeptide. In another example, the first recombinant host cell can have the genetic modification for reducing the production of one or more native enzymes that function to catabolize formate. In still a further embodiment, the genetic modification for reducing the production of one or more native enzymes that function to catabolize formate is a reduction in the expression of the gene encoding the FDH1 polypeptide and a reduction in the expression if the gene encoding the FDH2 polypeptide. In still another example, the first recombinant host cell can further lack the second genetic modification defined herein. In yet another embodiment, the first recombinant host cell is combined with a second recombinant host cell comprising the second generic modification defined herein. In an embodiment, the alpha-amylase variant has the amino acid sequence of SEQ ID NO: 1, SEQ ID NO: 2, SEQ ID NO: 7 or SEQ ID NO: 8.

According to a second aspect, the present disclosure provides a combination of (i) the first polypeptide having alpha-amylase activity as defined herein and (ii) a second polypeptide having glucoamylase activity on raw starch, wherein the second polypeptide is provided in a purified form or is expressed from a second recombinant host cell comprising a third genetic modification allowing the production of the second polypeptide. In an embodiment, the second recombinant host cell is a recombinant yeast host cell (e.g., from the genus Saccharomyces or from the species Saccharomyces cerevisiae). In another embodiment, the second polypeptide having glucoamylase activity is a glucoamylase polypeptide, a glucoamylase variant and/or a glucoamylase fragment and wherein:
the glucoamylase polypeptide has the amino acid sequence of SEQ ID NO: 5 or 6;
the glucoamylase variant has at least 70% identity with the glucoamylase polypeptide and has glucoamylase activity; and
the glucoamylase fragment has at least 70% identity with the glucoamylase polypeptide and has glucoamylase activity.

In a further embodiment, the second recombinant host cell further comprises a fourth genetic modification selected from the group consisting of a genetic modification for reducing the production of one or more native enzymes that function to produce glycerol or regulate glycerol synthesis and a genetic modification for reducing the production of one or more native enzymes that function to catabolize formate. For example, the second recombinant host cell can have the genetic modification for reducing the production of one or more native enzymes that function to produce glycerol or regulate glycerol synthesis. In an embodiment, the modification for reducing the production of one or more native enzymes that function to produce glycerol or regulate glycerol synthesis is a reduction in the expression of the gene encoding the GPD2 polypeptide. In yet another example, the second recombinant host cell can have the genetic modification for reducing the production of one or more native enzymes that function to catabolize formate. In a further embodiment, the genetic modification for reducing the production of one or more native enzymes that function to catabolize formate is a reduction in the expression of the gene encoding the FDH1 polypeptide and a reduction in the expression if the gene encoding the FDH2 polypeptide. In still another embodiment, the first polypeptide is expressed from the second recombinant host cell comprising the first genetic modification as herein.

According to a third aspect, the present disclosure concerns a population of recombinant host cells comprising the first recombinant host cell as defined herein and the second recombinant host cell as defined herein.

According to a fourth aspect, the present disclosure concerns a process for hydrolyzing starch in a raw form to make a fermentation product, the method comprising fermenting a medium with the first polypeptide as defined herein and the second polypeptide as defined herein or with the population defined herein. In an embodiment, the fermentation product is ethanol. In a further embodiment, the medium comprises raw starch. In yet another embodiment, the medium is derived from corn.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 compares the amylase activity of various Saccharomyces cerevisiae strains genetically engineered to express various α-amylases. The secreted amylase activity in gelatinized starch of a culture of wild-type (non-genetically modified) S. cerevisiae (M2390), a S. cerevisiae strain genetically engineered to express an α-amylase (MP85) from Bacillus amyloliquefaciens (MA) or a S. cerevisiae strain genetically engineered to express an α-amylase (MP98) from Saccharomycopsis fibuligera (MB) was determined. Results are shown as the absorbance at 540 nm in function of the S. cerevisiae strain.

FIGS. 2A and B compare the amylase activity of different combinations of purified α-amylases and glucoamylases on raw corn starch. (A) The secreted amylase activity on raw corn starch of a purified glucoamylase from Saccharomycopsis fibuligera (GLU0111, SEQ ID NO: 3 referred to as MP9 on FIG. 2A), a purified α-amylase from Bacillus amyloliquefaciens (AMYE, SEQ ID NO: 1, referred to as MP85 on FIG. 2A) or a combination of both MP9 and MP85 (in a purified form) was determined. Results are shown as the absorbance at 540 nm in function of the purified enzyme or combination of purified enzymes used. (B) The secreted amylase activity on raw starch of a purified glucoamylase from Saccharomycopsis fibuligera (GLU0111, SEQ ID NO: 3 referred to as MP9 on FIG. 2B), a purified α-amylase from Saccharomycopsis fibuligera (ALP1, SEQ ID NO: 2, also referred to as MP98 on FIG. 2) or a combination of both MP9 and MP98 (in a purified form) was determined. Results are shown as the absorbance at 540 nm in function of the purified enzyme or combination of purified enzymes used.

FIG. 3 shows the ethanol production during fermentation by S. cerevisiae strain M2390 of a fermentation substrate with a combination of purified MP9 and MP85 (in a weight ratio of 9:1). Results are shown as g/L of ethanol in function of supplemented enzymatic combination.

FIGS. 4A and B compare the enzymatic activity of various strains of S. cerevisiae on raw corn starch. (A) The secreted amylase activity on raw corn starch of a S. cerevisiae strain genetically engineered to express a glucoamylase from Saccharomycopsis fibuligera (M8841), a S. cerevisiae strain genetically engineered to co-express a glucoamylase from Saccharomycopsis fibuligera and an α-amylase from Bacillus amyloliquefaciens (MC) or a wild-type (non-genetically-modified) S. cerevisiae strain (M2390) was determined. Results are shown as the absorbance at 540 nm in function of the strain used. (B) The secreted amylase activity on raw corn starch of a S. cerevisiae strain genetically engineered to express a glucoamylase from Saccharomycopsis fibuligera (MP8841), a S. cerevisiae strain genetically engineered to express a glucoamylase from Saccharomycopsis fibuligera and an α-amylase from Saccharomycopsis fibuligera (MD) or a wild-type (non-genetically-modified) S. cerevisiae strain (M2390) was determined. Results are shown as the absorbance at 540 nm in function of the strain used.

### DETAILED DESCRIPTION

The present disclosure relates to the polypeptides having alpha-amylase activity for use in combination with polypeptides having glucoamylase activity to enhance the starch saccharification process (for example for improving the hydrolysis of starch, including the hydrolysis of raw starch). The polypeptides having alpha-amylase activity include, but are not limited to, polypeptides having the amino acid sequence of SEQ ID NO: 1 or 2, variants thereof (such as the polypeptides having the amino acid sequence of SEQ ID NO: 7 or 8) as well as fragments thereof. The polypeptides having alpha-amylase activity are intended to be used with or are combined with polypeptides having glucoamylase activity (such as, for example, the polypeptides having the amino acid sequence of SEQ ID NO: 5, variants thereof (such as polypeptides having the amino acid sequence of SEQ ID NO: 6) as well as fragments thereof). The use of such polypeptides, in some embodiments, limits the amount of enzymatic supplementation used during the fermentation process to achieve a similar amount of ethanol or increases the amount of ethanol produced.

When the polypeptides having the alpha-amylase activity and the polypeptides having the glucoamylase activity are expressed from heterologous nucleic acid molecules in one or more recombinant host cell capable of fermenting glucose to ethanol (such as, for example, in a recombinant yeast host cell), it allows for the break-down of starch to glucose, while simultaneously fermenting glucose to ethanol. In return, this balance between hydrolysis and fermentation keeps the presence of reducing sugars low and reduces the osmotic stress on the recombinant host cell. In addition to increasing process efficiency, recombinant expression of these distinct but complimentary enzymes is able to reduce the need for addition of expensive amylase mixtures, as well as reduce the need for the energy-intensive step of heating the raw material to temperatures approaching 180° C. (e.g., gelatinization) prior to fermentation.

### Polypeptides Having Alpha-Amylase Activity

Polypeptides having alpha-amylase activity (also referred to as alpha-amylases; EC 3.2.1.1) are endo-acting enzymes capable of hydrolyzing starch to maltose and maltodextrins. Alpha-amylases are calcium metalloenzymes which are unable to function in the absence of calcium. By acting at random locations along the starch chain, alpha-amylases break down long-chain carbohydrates, ultimately yielding maltotriose and maltose from amylose, or maltose, glucose and “limit dextrin” from amylopectin. Alpha-amylase activity can be determined by various ways by the person skilled in the art. For example, the alpha-amylase activity of a polypeptide can be determined directly by measuring the amount of reducing sugars generated by the polypeptide in an assay in which raw (corn) starch is used as the starting material. The alpha-amylase activity of a polypeptide can be measured indirectly by measuring the amount of reducing sugars generated by the polypeptide in an assay in which gelatinized (corn) starch is used as the starting material.

In the context of the present disclosure, the polypeptides having alpha-amylase activity can be derived from a bacteria, for example, from the genus Bacillus and, in some instances, from the species B. amyloliquefaciens. The polypeptides having alpha-amylase activity can be encoded by the amyE gene from B. amyloliquefaciens or an amyE gene ortholog. An embodiment of alpha-amylase polypeptide of the present disclosure is the AMYE polypeptide (GenBank Accession Number: ABS72727). The AMYE polypeptide comprises a catalytic domain (defined by amino acid residues located at positions 58 to 358) and an Aamy C domain (defined by amino acid residues located at positions 394 to 467). The AMYE polypeptide includes amino acid residues involved in the catalytic activity of the enzyme (e.g., active amino acid residues located at positions 99 to 100,103 to 104, 143, 146, 171, 215, 217 to 218, 220 to 221, 249, 251, 253, 309 to 310, 314) as well as amino acid residues involved in binding calcium (e.g., amino acid residues located at position 142, 187 and 212). In an embodiment, the polypeptides having alpha-amylase activity comprises both a catalytic domain and an AamyC domain of the AMYE polypeptide as indicated above. In still another embodiment, the polypeptides having alpha-amylase activity have one or more (and in some embodiments all) the amino acid residues indicated above involved in the catalytic and calcium binding activity of the AMYE polypeptide. It is possible to use a polypeptide which does not comprise its endogenous signal sequence, such as, for example, the amino acid sequence of SEQ ID NO: 2. In an embodiment, the nucleotide molecule encoding the AMYE polypeptide can include a signal sequence which is endogenous to the host cell expressing the nucleotide molecule. For example, when the host is S. cerevisiae, the nucleotide molecule encoding the AMYE polypeptide can include the signal sequence of a gene endogenously expressed in S. cerevisiae, such as the signal sequence of the invertase gene (SUC2), as shown in SEQ ID NO: 1.

In the context of the present disclosure, an “amyE gene ortholog” is understood to be a gene in a different species that evolved from a common ancestral gene by speciation. In the context of the present disclosure, an amyE ortholog retains the same function, e.g., it can act as an alpha-amylase. Known amyE gene orthologs include, but are not limited to those described at GenBank Accession numbers AGG59647.1 (B. subtilis), AHZ14317.1 (B. velezensis) and ACG63051.1 (Streptococcus equi), EFY01992 (Streptococcus dysgalactiae), EHI68955 (Streptococcus ictaluri), EFF68324 (Butyrivibrio crossotus), ADZ81868 (Clostridium lentocellum), AGX45116 (Clostridium saccharobutylicum), BAM49234 (Bacillus subtilis), ADP32662 (Bacillus atrophaeus), EFM08800 (Paenibacillus curdlanolyticus), EEP52889 (Clostridium butyricum) and COD81474 (Streptococcus pneumonia).

Still in the context of the present disclosure, the polypeptides having alpha-amylase activity include variants of the alpha-amylases polypeptides of SEQ ID NO: 1 or 2 (also referred to herein as alpha-amylase variants). A variant comprises at least one amino acid difference (substitution or addition) when compared to the amino acid sequence of the alpha-amylase polypeptide of SEQ ID NO: 1 or 2. In an embodiment, the alpha-amylase variants comprise both the catalytic domain and the AamyC domain of the AMYE polypeptide indicated above. In still another embodiment, the alpha-amylase variants have one or more (and in some embodiments all) the amino acid residues indicated above involved in the catalytic and calcium binding activity of the AMYE polypeptide. The alpha-amylase variants do exhibit alpha-amylase activity. In an embodiment, the variant alpha-amylase exhibits at least 50%, 60%, 70%, 80%, 90%, 95%, 96%, 97%, 98% or 99% of the alpha-amylase activity of the amino acid of SEQ ID NO: 2. The alpha-amylase variants also have at least 70%, 80%, 85%, 90%, 95%, 96%, 97%, 98% or 99% identity to the amino acid sequence of SEQ ID NO: 2. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant alpha-amylases described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant alpha-amylase can be also be a conservative variant or an allelic variant. As used herein, a conservative variant refers to alterations in the amino acid sequence that do not adversely affect the biological functions of the alpha amylase (e.g., hydrolysis of starch). A substitution, insertion or deletion is said to adversely affect the protein when the altered sequence prevents or disrupts a biological function associated with the alpha-amylase (e.g., the hydrolysis of starch into maltose and maltodextrins). For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the alpha-amylase.

In an embodiment, the alpha-amylase variant comprises the amino acid sequence of SEQ ID NO: 7 or 8. This alpha-amylase variant comprises a K N substitution at position 34 of SEQ ID NO: 1 (e.g., SEQ ID NO: 7) or at position 15 of SEQ ID NO: 2 (e.g., SEQ ID NO: 8).

The present disclosure also provide fragments of the alpha-amylases polypeptides and alpha-amylase variants described herein. A fragment comprises at least one less amino acid residue when compared to the amino acid sequence of the alpha-amylase polypeptide or variant and still possess the enzymatic activity of the full-length alpha-amylase. In an embodiment, the fragment of the alpha-amylase exhibits at least 50%, 60%, 70%, 80%, 90%, 95%, 96%, 97%, 98% or 99% of the alpha-amylase activity of the full-length amino acid of SEQ ID NO: 2. In an embodiment, the alpha-amylase fragments comprises both the catalytic domain and the AamyC domain of the AMYE polypeptide as indicated above. In still another embodiment, the alpha-amylase fragment has one or more (and in some embodiments all) the amino acid residues indicated above involved in the catalytic and calcium binding activity of the AMYE polypeptide. The alpha-amylase fragments can also have at least 70%, 80%, 85%, 90%, 95%, 96%, 97%, 98% or 99% identity to the amino acid sequence of SEQ ID NO: 1 or 2. The fragment can be, for example, a truncation of one or more amino acid residues at the amino-terminus, the carboxy terminus or both terminus of the alpha-amylase polypeptide or variant. Alternatively or in combination, the fragment can be generated from removing one or more internal amino acid residues. In an embodiment, the alpha-amylase fragment has at least 100, 150, 200, 250, 300, 350, 400, 450, 500, 550, 600, 650 or more consecutive amino acids of the alpha-amylase polypeptide or the variant.

The polypeptides having alpha-amylase activity can be provided in a (substantially) purified form. As used in the context of the present disclosure, the expression “purified form” refers to the fact that the polypeptides have been physically dissociated from at least one components required for their production (such as, for example, a host cell or a host cell fragment). A purified form of the polypeptide of the present disclosure can be a cellular extract of a host cell expressing the polypeptide being enriched for the polypeptide of interest (either through positive or negative selection). The expression “substantially purified form” refer to the fact that the polypeptides have been physically dissociated from the majority of components required for their production. In an embodiment, a polypeptide in a substantially purified form is at least 90%, 95%, 96%, 97%, 98% or 99% pure. Alternatively or in combination, the polypeptides having alpha-amylase activity can be provided by a recombinant host cell capable of expressing, in a recombinant fashion, the polypeptides.

### Polypeptides Having Glucoamylase Activity

In the context of the present disclosure, the polypeptides having alpha-amylase activity are intended to be used in combination with polypeptides having glucoamylase activity which exhibit hydrolytic activity against raw starch. As used in the context of the present disclosure, the term “raw starch” (which is also referred to native starch) refers to a substrate which has not been submitted to a heating/pH modifying step or to an alpha-amylase treatment step so as to denature the starch. Polypeptides having glucoamylase activity (also referred to as glucoamylases) are exo-acting enzymes capable of terminally hydrolyzing starch to glucose. Glucoamylase activity can be determined by various ways by the person skilled in the art. For example, the glucoamylase activity of a polypeptide can be determined directly by measuring the amount of reducing sugars generated by the polypeptide in an assay in which raw or gelatinized (corn) starch is used as the starting material.

In the context of the present disclosure, the polypeptides having glucoamylase activity can be derived from a yeast, for example, from the genus Saccharomycopsis and, in some instances, from the species S. fibuligera. The polypeptides having glucoamylase activity can be encoded by the glu0111 gene from S. fibuligera or a glu0111 gene ortholog. An embodiment of glucoamylase polypeptide of the present disclosure is the GLU0111 polypeptide (GenBank Accession Number: CAC83969.1). The GLU0111 polypeptide includes the following amino acids (or correspond to the following amino acids) which are associated with glucoamylase include, but are not limited to amino acids located at positions 41, 237, 470, 473, 479, 485, 487 of SEQ ID NO: 5. It is possible to use a polypeptide which does not comprise its endogenous signal sequence. In an embodiment, the polypeptides having glucoamylase activity include glucoamylases polypeptide comprising the amino acid sequence of SEQ ID NO: 5.

In the context of the present disclosure, a “glu0111 gene ortholog” is understood to be a gene in a different species that evolved from a common ancestral gene by speciation. In the context of the present disclosure, a glu0111 ortholog retains the same function, e.g., it can act as a glucoamylase. Glu0111 gene orthologs includes but are not limited to, the nucleic acid sequence of GenBank Accession Number XP_003677629.1 (Naumovozyma castelth) XP_003685231.1 (Tetrapisispora phaffii), XP_455264.1 (Kluyveromyces lactis), XP_446481.1 (Candida glabrata), EER33360.1 (Candida tropicalis), EEQ36251.1 (Clavispora lusitaniae), ABN68429.2 (Scheffersomyces stipitis), AAS51695.2 (Eremothecium gossypii), EDK43905.1 (Lodderomyces elongisporus), XP_002555474.1 (Lachancea thermotolerans), EDK37808.2 (Pichia guilliermondii), CAA86282 (Saccharomyces cerevisiae), XP_003680486.1 (Torulaspora delbrueckii), XP_503574.1 (Yarrowia lipolytica), XP_002496552.1 (Zygosaccharomyces rouxii), CAX42655.1 (Candida dubliniensis), XP_002494017.1 (Komagataella pastoris) and AET38805.1 (Eremothecium cymbalariae).

Still in the context of the present disclosure, the polypeptides having glucoamylase activity include variants of the glucoamylases polypeptides of SEQ ID NO: 5 (also referred to herein as glucoamylase variants). A variant comprises at least one amino acid difference (substitution or addition) when compared to the amino acid sequence of the glucoamylase polypeptide of SEQ ID NO: 5. The glucoamylase variants do exhibit glucoamylase activity. In an embodiment, the variant glucoamylase exhibits at least 50%, 60%, 70%, 80%, 90%, 95%, 96%, 97%, 98% or 99% of the glucoamylase activity of the amino acid of SEQ ID NO: 5. The glucoamylase variants also have at least 70%, 80%, 85%, 90%, 95%, 96%, 97%, 98% or 99% identity to the amino acid sequence of SEQ ID NO: 5. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant glucoamylases described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant glucoamylase can also be a conservative variant or an allelic variant. As used herein, a conservative variant refers to alterations in the amino acid sequence that do not adversely affect the biological functions of the glucoamylase. A substitution, insertion or deletion is said to adversely affect the protein when the altered sequence prevents or disrupts a biological function associated with the glucoamylase (e.g., the hydrolysis of starch into glucose). For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the glucoamylase.

In an embodiment, the glucoamylase variant has the amino acid sequence of SEQ ID NO: 6.

The present disclosure also provide fragments of the glucoamylases polypeptides and glucoamylase variants described herein. A fragment comprises at least one less amino acid residue when compared to the amino acid sequence of the glucoamylase polypeptide or variant and still possess the enzymatic activity of the full-length glucoamylase. In an embodiment, the glucoamylase fragment exhibits at least 50%, 60%, 70%, 80%, 90%, 95%, 96%, 97%, 98% or 99% of the full-length glucoamylase of the amino acid of SEQ ID NO: 5. The glucoamylase fragments can also have at least 70%, 80%, 85%, 90%, 95%, 96%, 97%, 98% or 99% identity to the amino acid sequence of SEQ ID NO: 5. The fragment can be, for example, a truncation of one or more amino acid residues at the amino-terminus, the carboxy terminus or both termini of the glucoamylase polypeptide or variant. Alternatively or in combination, the fragment can be generated from removing one or more internal amino acid residues. In an embodiment, the glucoamylase fragment has at least 100, 150, 200, 250, 300, 350, 400, 450, 500 or more consecutive amino acids of the glucoamylase polypeptide or the variant.

Embodiments of polypeptides having glucoamylase activity have been also been described in PCT/US2012/032443 (published under WO/2012/138942) and PCT/US2011/039192 (published under WO/2011/153516) can also be used in the context of the present disclosure.

The polypeptides having glucoamylase activity, their fragments and their variants exhibit enzymatic activity towards raw starch. The GLU0111 polypeptide presented herein as well as glucomylases from Rhizopus oryzae and Corticium rolfsiiare are known to exhibit enzymatic activity towards raw starch.

The polypeptides having glucoamylase activity can be provided in a (substantially) purified form. As used in the context of the present disclosure, the expression “purified form” refer to the fact that the polypeptides have been physically dissociated from at least one components required for their production (a host cell or a host cell fragment). A purified form of the polypeptide of the present disclosure can be a cellular extract of a host cell expressing the polypeptide being enriched for the polypeptide of interest (either by positive or negative selection). The expression “substantially purified form” refer to the fact that the polypeptides have been physically dissociated from the majority of components required for their production. In an embodiment, a polypeptide in a substantially purified form is at least 90%, 95%, 96%, 97%, 98% or 99% pure. Alternatively or in combination, the polypeptides having glucoamylase activity can be provided by a recombinant host cell capable of expressing, in a recombinant fashion, the polypeptides.

### Recombinant Host Cells

The polypeptides described herein can independently be provided in a purified form or expressed in a recombinant host cell (e.g., the same or different recombinant host cells). The recombinant host cell includes at least one genetic modification. In the context of the present disclosure, when recombinant yeast cell is qualified has “having a genetic modification” or as being “genetically engineered”, it is understood to mean that it has been manipulated to either add at least one or more heterologous or exogenous nucleic acid residue and/or remove at least one endogenous (or native) nucleic acid residue. The genetic manipulations did not occur in nature and is the results of in vitro manipulations of the recombinant host cell. When the genetic modification is the addition of a heterologous nucleic acid molecule, such addition can be made once or multiple times at the same or different integration sites. When the genetic modification is the modification of an endogenous nucleic acid molecule, it can be made in one or both copies of the targeted gene.

When expressed in a recombinant host, the polypeptides described herein are encoded on one or more heterologous nucleic acid molecule. The term “heterologous” when used in reference to a nucleic acid molecule (such as a promoter or a coding sequence) refers to a nucleic acid molecule that is not natively found in the recombinant host cell. “Heterologous” also includes a native coding region, or portion thereof, that is removed from the source organism and subsequently reintroduced into the source organism in a form that is different from the corresponding native gene, e.g., not in its natural location in the organism's genome. The heterologous nucleic acid molecule is purposively introduced into the recombinant host cell. The term “heterologous” as used herein also refers to an element (nucleic acid or protein) that is derived from a source other than the endogenous source. Thus, for example, a heterologous element could be derived from a different strain of host cell, or from an organism of a different taxonomic group (e.g., different kingdom, phylum, class, order, family genus, or species, or any subgroup within one of these classifications). The term “heterologous” is also used synonymously herein with the term “exogenous”.

When a heterologous nucleic acid molecule is present in the recombinant host cell, it can be integrated in the host cell's genome. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies in the yeast host cell's genome. Alternatively, the heterologous nucleic acid molecule can be independently replicating from the yeast's genome. In such embodiment, the nucleic acid molecule can be stable and self-replicating.

In the context of the present disclosure, the recombinant host cell can be a recombinant yeast host cell. Suitable recombinant yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, S. cerevisiae, S. bulderi, S. barnetti, S. exiguus, S. uvarum, S. diastaticus, K. lactis, K. marxianus or K. fragilis. In some embodiments, the recombinant yeast host cell is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In some embodiment, the recombinant host cell can be an oleaginous yeast cell. For example, the recombinant oleaginous yeast host cell can be from the genera Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the recombinant host cell can be an oleaginous microalgae host cell (e.g., for example, from the genera Thraustochytrium or Schizochytrium). In an embodiment, the recombinant yeast host cell is from the genus Saccharomyces and, in some embodiments, from the species Saccharomyces cerevisiae. In one particular embodiment, the recombinant yeast host cell is Saccharomyces cerevisiae.

One of the genetic modification that can be introduced into the recombinant host is the introduction of one or more of an heterologous nucleic acid molecule encoding an heterologous polypeptide (such as, for example, the polypeptides having alpha-amylase activity as described herein).

In a first embodiment, the recombinant host cell comprise a first genetic modification (e.g., a first heterologous nucleic acid molecule) allowing the recombinant expression of the polypeptide having alpha-amylase activity. In such embodiment, a heterologous nucleic acid molecule encoding the polypeptide having alpha-amylase activity can be introduced in the recombinant host to express the polypeptide having alpha-amylase activity. The expression of the polypeptide having alpha-amylase activity can be constitutive or induced.

The recombinant host cell comprising the first genetic modification can also include a further (second) genetic modification for reducing the production of one or more native enzymes that function to produce glycerol or regulate glycerol synthesis, for allowing the production of the second polypeptide having glucoamylase activity and/or for reducing the production of one or more native enzymes that function to catabolize formate. Alternatively, the recombinant host cell comprising the first genetic modification be used in combination with a further recombinant host cell which includes a further (second) genetic modification for reducing the production of one or more native enzymes that function to produce glycerol or regulate glycerol synthesis, for allowing the production of the second polypeptide having glucoamylase activity and/or for reducing the production of one or more native enzymes that function to catabolize formate. As used in the context of the present disclosure, the expression “reducing the production of one or more native enzymes that function to produce glycerol or regulate glycerol synthesis” refers to a genetic modification which limits or impedes the expression of genes associated with one or more native polypeptides (in some embodiments enzymes) that function to produce glycerol or regulate glycerol synthesis, when compared to a corresponding host strain which does not bear the second genetic modification. In some instances, the second genetic modification reduces but still allows the production of one or more native polypeptides that function to produce glycerol or regulate glycerol synthesis. In other instances, the second genetic modification inhibits the production of one or more native enzymes that function to produce glycerol or regulate glycerol synthesis. In some embodiments, the recombinant host cells bear a plurality of second genetic modifications, wherein at least one reduces the production of one or more native polypeptides and at least another inhibits the production of one or more native polypeptides.

Alternatively, the recombinant host cell comprising the first genetic modification can also exclude a further (second) genetic modification for reducing the production of one or more native enzymes that function to produce glycerol or regulate glycerol synthesis, for allowing the production of the second polypeptide having glucoamylase activity and/or for reducing the production of one or more native enzymes that function to catabolize formate. In such embodiment, the recombinant host cell can be combined with a further (second) recombinant yeast host cells comprising the further (second) genetic modification.

As used in the context of the present disclosure, the expression “native polypeptides that function to produce glycerol or regulate glycerol synthesis” refers to polypeptides which are endogenously found in the recombinant host cell. Native enzymes that function to produce glycerol include, but are not limited to, the GPD1 and the GPD2 polypeptide (also referred to as GPD1 and GPD2 respectively). Native enzymes that function to regulate glycerol synthesis include, but are not limited to, the FPS1 polypeptide. In an embodiment, the recombinant host cell bears a genetic modification in at least one of the gpd1 gene (encoding the GPD1 polypeptide), the gpd2 gene (encoding the GPD2 polypeptide), the fps1 gene (encoding the FPS1 polypeptide) or orthologs thereof. In another embodiment, the recombinant yeast host cell bears a genetic modification in at least two of the gpd1 gene (encoding the GPD1 polypeptide), the gpd2 gene (encoding the GPD2 polypeptide), the fps1 gene (encoding the FPS1 polypeptide) or orthologs thereof. In still another embodiment, the recombinant yeast host cell bears a genetic modification in each of the gpd1 gene (encoding the GPD1 polypeptide), the gpd2 gene (encoding the GPD2 polypeptide) and the fps1 gene (encoding the FPS1 polypeptide) or orthologs thereof. Examples of recombinant yeast host cells bearing such genetic modification(s) leading to the reduction in the production of one or more native enzymes that function to produce glycerol or regulate glycerol synthesis are described in WO 2012/138942. Preferably, the recombinant host cell has a genetic modification (such as a genetic deletion or insertion) only in one enzyme that functions to produce glycerol, in the gpd2 gene, which would cause the host cell to have a knocked-out gpd2 gene. In some embodiments, the recombinant host cell can have a genetic modification in the gpd1 gene, the gpd2 gene and the fps1 gene resulting is a recombinant host cell being knock-out for the gpd1 gene, the gpd2 gene and the fps1 gene.

As used in the context of the present disclosure, the expression “for reducing the production of one or more native enzymes that function to catabolize formate”. As used in the context of the present disclosure, the expression “native polypeptides that function to catabolize formate” refers to polypeptides which are endogenously found in the recombinant host cell. Native enzymes that function to catabolize formate include, but are not limited to, the FDH1 and the FDH2 polypeptides (also referred to as FDH1 and FDH2 respectively). In an embodiment, the recombinant yeast host cell bears a genetic modification in at least one of the fdh1 gene (encoding the FDH1 polypeptide), the fdh2 gene (encoding the FDH2 polypeptide) or orthologs thereof. In another embodiment, the recombinant yeast host cell bears genetic modifications in both the fdh1 gene (encoding the FDH1 polypeptide) and the fdh2 gene (encoding the FDH2 polypeptide) or orthologs thereof. Examples of recombinant yeast host cells bearing such genetic modification(s) leading to the reduction in the production of one or more native enzymes that function to catabolize formate are described in WO 2012/138942. Preferably, the recombinant yeast host cell has genetic modifications (such as a genetic deletion or insertion) in the fdh1 gene and in the fdh2 gene which would cause the host cell to have knocked-out fdh1 and fdh2 genes.

In some embodiments, the nucleic acid molecules encoding the heterologous polypeptides, fragments or variants that can be introduced into the recombinant host cells are codon-optimized with respect to the intended recipient recombinant host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules of the present disclosure comprise a coding region for the heterologous polypeptide. A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The nucleic acid molecules described herein can comprise transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

The heterologous nucleic acid molecule can be introduced in the host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

In the heterologous nucleic acid molecule described herein, the promoter and the nucleic acid molecule coding for the heterologous polypeptide are operatively linked to one another. In the context of the present disclosure, the expressions “operatively linked” or “operatively associated” refers to fact that the promoter is physically associated to the nucleotide acid molecule coding for the heterologous polypeptide in a manner that allows, under certain conditions, for expression of the heterologous protein from the nucleic acid molecule. In an embodiment, the promoter can be located upstream (5′) of the nucleic acid sequence coding for the heterologous protein. In still another embodiment, the promoter can be located downstream (3′) of the nucleic acid sequence coding for the heterologous protein. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the heterologous protein. The promoters can be located, in view of the nucleic acid molecule coding for the heterologous protein, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease Si), as well as protein binding domains (consensus sequences) responsible for the binding of the polymerase.

The promoter can be heterologous to the nucleic acid molecule encoding the heterologous polypeptide. The promoter can be heterologous or derived from a strain being from the same genus or species as the recombinant host cell. In an embodiment, the promoter is derived from the same genus or species of the yeast host cell and the heterologous polypeptide is derived from different genera that the host cell.

The recombinant host cell can be further genetically modified to allow for the production of additional heterologous polypeptides. In an embodiment, the recombinant yeast host cell can be used for the production of an enzyme, and especially an enzyme involved in the cleavage or hydrolysis of its substrate (e.g., a lytic enzyme and, in some embodiments, a saccharolytic enzyme). In still another embodiment, the enzyme can be a glycoside hydrolase. In the context of the present disclosure, the term “glycoside hydrolase” refers to an enzyme involved in carbohydrate digestion, metabolism and/or hydrolysis, including amylases (other than those described above), cellulases, hemicellulases, cellulolytic and amylolytic accessory enzymes, inulinases, levanases, trehalases, pectinases, and pentose sugar utilizing enzymes. In another embodiment, the enzyme can be a protease. In the context of the present disclosure, the term “protease” refers to an enzyme involved in protein digestion, metabolism and/or hydrolysis. In yet another embodiment, the enzyme can be an esterase. In the context of the present disclosure, the term “esterase” refers to an enzyme involved in the hydrolysis of an ester from an acid or an alcohol, including phosphatases such as phytases.

The additional heterologous polypeptide can be an “amylolytic enzyme”, an enzyme involved in amylase digestion, metabolism and/or hydrolysis. The term “amylase” refers to an enzyme that breaks starch down into sugar. All amylases are glycoside hydrolases and act on α-1,4-glycosidic bonds. Some amylases, such as γ-amylase (glucoamylase), also act on α-1,6-glycosidic bonds. Amylase enzymes include α-amylase (EC 3.2.1.1), β-amylase (EC 3.2.1.2), and γ-amylase (EC 3.2.1.3). The α-amylases are calcium metalloenzymes, unable to function in the absence of calcium. By acting at random locations along the starch chain, α-amylase breaks down long-chain carbohydrates, ultimately yielding maltotriose and maltose from amylose, or maltose, glucose and “limit dextrin” from amylopectin. Because it can act anywhere on the substrate, α-amylase tends to be faster-acting than β-amylase. Another form of amylase, β-amylase is also synthesized by bacteria, fungi, and plants. Working from the non-reducing end, β-amylase catalyzes the hydrolysis of the second α-1,4 glycosidic bond, cleaving off two glucose units (maltose) at a time. Another amylolytic enzyme is α-glucosidase that acts on maltose and other short malto-oligosaccharides produced by α-, β-, and γ-amylases, converting them to glucose. Another amylolytic enzyme is pullulanase. Pullulanase is a specific kind of glucanase, an amylolytic exoenzyme, that degrades pullulan. Pullulan is regarded as a chain of maltotriose units linked by alpha-1,6-glycosidic bonds. Pullulanase (EC 3.2.1.41) is also known as pullulan-6-glucanohydrolase (debranching enzyme). Another amylolytic enzyme, isopullulanase, hydrolyses pullulan to isopanose (6-alpha-maltosylglucose). Isopullulanase (EC 3.2.1.57) is also known as pullulan 4-glucanohydrolase. An “amylase” can be any enzyme involved in amylase digestion, metabolism and/or hydrolysis, including α-amylase, β-amylase, glucoamylase, pullulanase, isopullulanase, and alpha-glucosidase.

The additional heterologous polypeptide can be a “cellulolytic enzyme”, an enzyme involved in cellulose digestion, metabolism and/or hydrolysis. The term “cellulase” refers to a class of enzymes that catalyze cellulolysis (i.e., the hydrolysis) of cellulose. Several different kinds of cellulases are known, which differ structurally and mechanistically. There are general types of cellulases based on the type of reaction catalyzed: endocellulase breaks internal bonds to disrupt the crystalline structure of cellulose and expose individual cellulose polysaccharide chains; exocellulase cleaves 2-4 units from the ends of the exposed chains produced by endocellulase, resulting in the tetrasaccharides or disaccharide such as cellobiose. There are two main types of exocellulases (or cellobiohydrolases, abbreviate CBH)—one type working processively from the reducing end, and one type working processively from the non-reducing end of cellulose; cellobiase or beta-glucosidase hydrolyses the exocellulase product into individual monosaccharides; oxidative cellulases that depolymerize cellulose by radical reactions, as for instance cellobiose dehydrogenase (acceptor); cellulose phosphorylases that depolymerize cellulose using phosphates instead of water. In the most familiar case of cellulase activity, the enzyme complex breaks down cellulose to beta-glucose. A “cellulase” can be any enzyme involved in cellulose digestion, metabolism and/or hydrolysis, including an endoglucanase, glucosidase, cellobiohydrolase, xylanase, glucanase, xylosidase, xylan esterase, arabinofuranosidase, galactosidase, cellobiose phosphorylase, cellodextrin phosphorylase, mannanase, mannosidase, xyloglucanase, endoxylanase, glucuronidase, acetylxylanesterase, arabinofuranohydrolase, swollenin, glucuronyl esterase, expansin, pectinase, and feruoyl esterase protein.

The additional heterologous polypeptide can have “hemicellulolytic activity”, an enzyme involved in hemicellulose digestion, metabolism and/or hydrolysis. The term “hemicellulase” refers to a class of enzymes that catalyze the hydrolysis of cellulose. Several different kinds of enzymes are known to have hemicellulolytic activity including, but not limited to, xylanases and mannanases.

The additional heterologous polypeptide can have “xylanolytic activity”, an enzyme having the is ability to hydrolyze glycosidic linkages in oligopentoses and polypentoses. The term “xylanase” is the name given to a class of enzymes which degrade the linear polysaccharide beta-1,4-xylan into xylose, thus breaking down hemicellulose, one of the major components of plant cell walls. Xylanases include those enzymes that correspond to Enzyme Commission Number 3.2.1.8. The heterologous protein can also be a “xylose metabolizing enzyme”, an enzyme involved in xylose digestion, metabolism and/or hydrolysis, including a xylose isomerase, xylulokinase, xylose reductase, xylose dehydrogenase, xylitol dehydrogenase, xylonate dehydratase, xylose transketolase, and a xylose transaldolase protein. A “pentose sugar utilizing enzyme” can be any enzyme involved in pentose sugar digestion, metabolism and/or hydrolysis, including xylanase, arabinase, arabinoxylanase, arabinosidase, arabinofuranosidase, arabinoxylanase, arabinosidase, and arabinofuranosidase, arabinose isomerase, ribulose-5-phosphate 4-epimerase, xylose isomerase, xylulokinase, xylose reductase, xylose dehydrogenase, xylitol dehydrogenase, xylonate dehydratase, xylose transketolase, and/or xylose transaldolase.

The additional heterologous polypeptide can have “mannanic activity”, an enzyme having the ability to hydrolyze the terminal, non-reducing β-D-mannose residues in β-D-mannosides. Mannanases are capable of breaking down hemicellulose, one of the major components of plant cell walls. Xylanases include those enzymes that correspond to Enzyme Commission Number 3.2.25.

The additional heterologous polypeptide can be a “pectinase”, an enzyme, such as pectolyase, pectozyme and polygalacturonase, commonly referred to in brewing as pectic enzymes. These enzymes break down pectin, a polysaccharide substrate that is found in the cell walls of plants.

The additional heterologous polypeptide can have “phytolytic activity”, an enzyme catalyzing the conversion of phytic acid into inorganic phosphorus. Phytases (EC 3.2.3) can be belong to the histidine acid phosphatases, β-propeller phytases, purple acid phosphastases or protein tyrosine phosphatase-like phytases family.

The additional heterologous polypeptide can have “proteolytic activity”, an enzyme involved in protein digestion, metabolism and/or hydrolysis, including serine proteases, threonine proteases, cysteine proteases, aspartate proteases, glutamic acid proteases and metalloproteases.

### Combination of Polypeptides Having Alpha-Amylase Activity and Having Glucoamylase Activity

As indicated above, the polypeptides having alpha-amylases activity are intended to be combined with polypeptides having glucoamylase activity to improve saccharification. In some embodiments, and as shown in the Example below, the combination of polypeptides having alpha-amylase activity and of polypeptides having glucoamylase activity exhibit a synergistic effect with respect to the hydrolysis of starch (e.g., hydrolysis rate), particularly of the hydrolysis of starch in a raw (non-gelatinized) form, which ultimately favors the production of ethanol.

In an embodiment, the polypeptides having alpha-amylase activity are used in a substantially purified form in combination with the polypeptides having glucoamylase activity. In such embodiment, the substantially purified polypeptides having alpha-amylase activity can be used to supplement a fermentation medium comprising starch and a microorganism capable of fermenting glucose into ethanol (“fermentation microorganism”). Still in such embodiment, the source of the polypeptides having alpha-amylase activity can be provided exclusively from the substantially purified polypeptides having alpha-amylase activity, or in combination with a recombinant host cell, to be included in the fermentation medium, expressing the polypeptides having alpha-amylase activity in a recombinant fashion. The polypeptides having glucoamylase activity can be provided, in the fermentation medium, in a substantially purified form and/or expressed from a recombinant host cell in a recombinant fashion. The recombinant host cell (expressing the polypeptides having alpha-amylase activity and/or the polypeptides having glucoamylase activity) can be the fermentation microorganism. In still a further embodiment, when the polypeptides having alpha-amylase activity are provided, in the fermentation medium, in a substantially purified form, the polypeptides having glucoamylase activity are expressed, in the fermentation medium, from a recombinant host cell in a recombinant fashion. In yet another embodiment, the only enzymatic supplementation that is used when the polypeptides having glucoamylase activity are expressed from a recombinant host is the polypeptide having alpha-amylase activity as described herein (e.g., no additional exogenous amylolytic enzymes are added to the fermentation medium).

In an embodiment, the polypeptides having alpha-amylase activity can be expressed from a recombinant host cell in a recombinant fashion in combination with the polypeptides having glucoamylase activity. In such embodiment, the recombinant host cell expressing the polypeptides having alpha-amylase activity are added to a fermentation medium comprising starch. If the recombinant host expressing the polypeptides having alpha-amylase activity is capable of fermenting glucose into ethanol, then no additional fermentation microorganism is required (but can nevertheless be added). However, if the recombinant host expressing the polypeptides having alpha-amylase activity is not capable of fermentation glucose into ethanol, then it is necessary to include a fermentation organism capable of fermenting glucose into ethanol in the fermentation medium. Still in such embodiment, in the fermentation medium, the source of the polypeptides having alpha-amylase activity can be provided exclusively from recombinant host cell expressing the polypeptides having alpha-amylase activity in a recombinant fashion or in combination with the substantially purified polypeptides having alpha-amylase activity. In this embodiment, the polypeptides having glucoamylase activity can be provided, in the fermentation medium, in a substantially purified form and/or expressed from a recombinant host cell in a recombinant fashion. The recombination host cell (expressing the polypeptides having alpha-amylase activity and/or the polypeptides having glucoamylase activity) can be the fermentation microorganism. In still a further embodiment, when the polypeptides having alpha-amylase activity are expressed, in the fermentation medium, from a recombinant host cell in a recombinant fashion, the polypeptides having glucoamylase activity are expressed, in the fermentation medium, from the same or a different recombinant host cell in a recombinant fashion. In yet another embodiment, when both the polypeptides having alpha-amylase activity and having glucoamylase activity are expressed from a recombinant source (the same or different) no additional exogenous amylolytic enzyme is included in the fermentation medium during the fermentation.

As indicated herein the recombinant host cells described herein can include additional modifications that those necessary to allow the expression of the polypeptides having alpha-amylase activity and/or the polypeptides having glucoamylase activity.

The present application also provides a population of recombinant host cells expressing the polypeptides having alpha-amylase activity to be combined with polypeptides having glucoamylase activity. In an embodiment, the population of host cells is homogeneous, i.e., each recombinant host cell of the population comprises the same genetic modifications allowing for the expression of the polypeptides having alpha-amylase activity. For example, the homogeneous population of cells can comprise recombinant host cells expressing the polypeptides having alpha-amylase activity and can optionally further express the polypeptides having glucoamylase activity. In yet another example, the homogenous population of cells can comprise recombinant host cells expressing the polypeptides having alpha-amylase activity in combination with polypeptides having glucoamylase activity in a substantially purified form.

In another embodiment, the population of host cells is heterogeneous, i.e., the population comprises two or more subpopulations of recombinant host cells wherein each members of the same subpopulation of recombinant host cells comprises at least one common genetic modification(s) which differ from the at least other common genetic modification(s) shared amongst the other subpopulation of recombinant cells. For example, in the heterogeneous population of recombinant cells, the first subpopulation of recombinant cells can include a genetic modification allowing for the expression of the polypeptides having alpha-amylase activity but not for the polypeptides having glucoamylase activity while the second subpopulations of recombinant cells include a genetic modification allowing for the expression of the polypeptides having glucoamylase activity but not for the polypeptides having alpha-amylase activity. In such embodiment, the second subpopulation of cells can include additional genetic modification, for example, a genetic modification for reducing the production of one or more native enzymes that function to produce glycerol or regulate glycerol synthesis and/or a genetic modification for reducing the production of one or more native enzymes that function to catabolize formate.

In the embodiment in which the heterogeneous population comprises a first subpopulation expressing the polypeptides having alpha-amylase activity and a second subpopulation expressing the polypeptides having glucoamylase activity. In such embodiment, at the start of the fermentation, the ratio of the secreted alpha-amylase to glucoamylase, in a fermentation medium which has not been supplemented with a purified enzymatic preparation, is about 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 or 1:20.

### Process for Hydrolyzing Lignocellulosic Material

The polypeptides and recombinant host cells described herein can be used to hydrolyze (e.g., saccharify) starch into glucose to allow a concomitant or subsequent fermentation of glucose into ethanol. If the polypeptides can be used in a substantially purified form as an additive to a fermentation process. Alternatively or in combination, the polypeptides can be expressed from one or more recombinant host cell during the fermentation process.

The process comprises combining a substrate to be hydrolyzed (optionally included in a fermentation medium) with the recombinant host cells expressing the polypeptides and/or with the polypeptides in a substantially purified form. In an embodiment, the substrate to be hydrolyzed is a lignocellulosic biomass and, in some embodiments, it comprises starch (in a gelatinized or raw form). In some embodiments, the use of recombinant host cells or the purified polypeptides limits or avoids the need of adding additional external source of purified enzymes during fermentation to allow the breakdown of starch. The expression of the polypeptides in a recombinant host cell is advantageous because it can reduce or eliminate the need to supplement the fermentation medium with external source of purified enzymes (e.g., glucoamylase and/or alpha-amylase) while allowing the fermentation of the lignocellulosic biomass into a fermentation product (such as ethanol).

The polypeptides having alpha-amylase activity described herein can be used to increase the production of a fermentation product during fermentation. The process comprises combining a substrate to be hydrolyzed (optionally included in a fermentation medium) with the polypeptide having alpha-amylase activity (either in a purified form or expressed in a recombinant host cell) and the polypeptide having glucoamylase activity (either in a purified form or expression in a recombinant host cell). In an embodiment, the process can comprise combining the substrate with an heterologous population of recombinant host cells as described herein. In an embodiment, the substrate to be hydrolyzed is a lignocellulosic biomass and, in some embodiments, it comprises starch (in a gelatinized or raw form). In still another embodiment, the substrate comprises raw starch and the process excludes the step of heating (gelatinizing) the starch prior to fermentation and/or the step of adding other enzymes, such as other alpha-amylases, than those described herein. This embodiment is advantageous because it can reduce or eliminate the need to supplement the fermentation medium with external source of purified enzymes (e.g., glucoamylase and/or alpha-amylase) while allowing the fermentation of the lignocellulosic biomass into a fermentation product (such as ethanol). However, in some circumstances, it may be advisable to supplement the medium with a polypeptide having alpha-amylase activity in a purified form. Such polypeptide can be produced in a recombinant fashion in a recombinant host cell.

The production of ethanol can be performed at temperatures of at least about 25° C., about 28° C., about 30° C., about 31° C., about 32° C., about 33° C., about 34° C., about 35° C., about 36° C., about 37° C., about 38° C., about 39° C., about 40° C., about 41° C., about 42° C., or about 50° C. In some embodiments, when a thermotolerant yeast cell is used in the process, the process can be conducted at temperatures above about 30° C., about 31° C., about 32° C., about 33° C., about 34° C., about 35° C., about 36° C., about 37° C., about 38° C., about 39° C., about 40° C., about 41° C., about 42° C., or about 50° C.

In some embodiments, the process can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, or at least about 500 mg per hour per liter. Ethanol production can be measured using any method known in the art.

For example, the quantity of ethanol in fermentation samples can be assessed using HPLC analysis. Many ethanol assay kits are commercially available that use, for example, alcohol oxidase enzyme based assays.

The present disclosure will be more readily understood by referring to the following examples which are given to illustrate the invention rather than to limit its scope.

### Example

TABLE 1
Description of the Enzymes Used in the Example

| Designation | Description |
|---|---|
| MP85 | Native alpha-amylase from Bacillus amyloliquefaciens |
| | (Accession Number ABS72727) |
| | Protein with signal sequence of S. cerevisia invertase |
| | (SUC2) gene shown as SEQ ID NO: 1 |
| | Protein without signal sequence shown as SEQ ID NO: 2 |
| MP98 | Native alpha-amylase from Saccharomycopsis fibuligera |
| | (Accession Number CAA29233.1) |
| | Protein with signal sequence SEQ ID NO: 3 |
| | Protein without signal sequence SEQ ID NO: 4 |
| MP9 | Native glucoamylase from Saccharomycopsis fibuligera |
| | (Accession Number CAC83969.1) |
| | Protein listed as SEQ ID NO: 5 |
| MP743 | Mutant MP9 (A40N) |
| | Protein listed as SEQ ID NO: 6 |
| MP775 | Mutant MP85 (K34N) |
| | Protein listed as SEQ ID NO: 7 |

TABLE 2
Description of the S. cerevisiae Strains Presented in the Example

| | α-amylase | Other transgenes | Genes |
|---|---|---|---|
| Designation | expressed | expressed | inactivated |
| M2390 | None | None | |
| (wild-type, | | | |
| control) | | | |
| M8841 | | gene encoding MP9 | Δgpd2 |
| | | pfla | Δfdh1 |
| | | pflb | Δfdh2 |
| | | adhe | Δfcy1 |
| M10156 | gene encoding MP775 | gene encoding MP9 | Δgpd2 |
| | | pfla | Δfdh1 |
| | | pflb | Δfdh2 |
| | | adhe | Δfcy1 |
| M10624 | gene encoding MP775 | gene encoding MP743 | Δgpd2 |
| | | pfla | Δfdh1 |
| | | pflb | Δfdh2 |
| | | adhe | Δfcy1 |
| MA | gene encoding MP85 | None | None |
| MB | gene encoding MP98 | None | None |
| MC | gene encoding MP85 | gene encoding MP9 | Δgpd2 |
| | | pfla | Δfdh1 |
| | | pflb | Δfdh2 |
| | | adhe | Δfcy1 |
| MD | gene encoding MP98 | gene encoding MP9 | Δgpd2 |
| | | pfla | Δfdh1 |
| | | pflb | Δfdh2 |
| | | adhe | Δfcy1 |

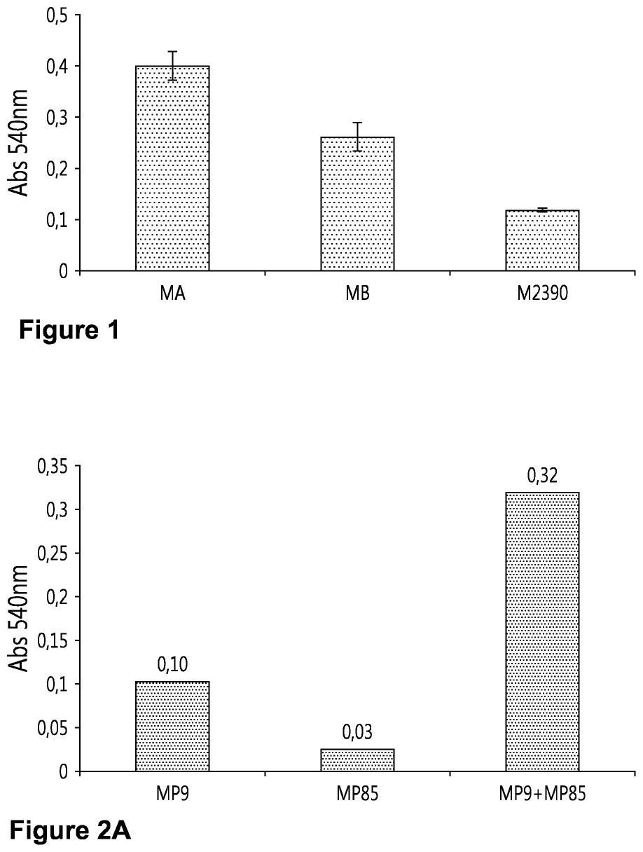

The gene coding for alpha-amylases from Bacillus amyloliquefaciens (amyE gene coding for MP85) and Saccharomycopsis fibuligera (alp1 gene coding for MP98) were codon optimized and cloned into Saccharomyces cerevisiae under regulation of the highly constitutive TEF2 promoter. The secreted amylase activity of each strains was measured using a plate-based starch assay. Briefly, strains of interest were grown 24-72 h in YPD. The cultures were then centrifuged at 3000 rpm to separate the cells from the culture supernatant containing the secreted enzymes. The supernatant was then added to a 1% cornstarch solution in a 50 mM sodium acetate buffer (pH 5.0). For the gelatinized starch assay, the corn starch solution was heated at 99° C. for 5 mins. For raw starch assays, the heating step was not included. The assay was conducted using a 4:1 starch solution:supernatant ratio and incubated at 35° C. for 1-4 h. The reducing sugars were measured using the Dinitrosalicylic Acid Reagent Solution (DNS) method, using a 2:1 DNS:starch assay ratio and boiled at 100° C. for 5 mins. The absorbance was measured at 540 nm. As shown on FIG. 1, both genetically-engineered strains exhibited amylase activity on gelatinized starch.

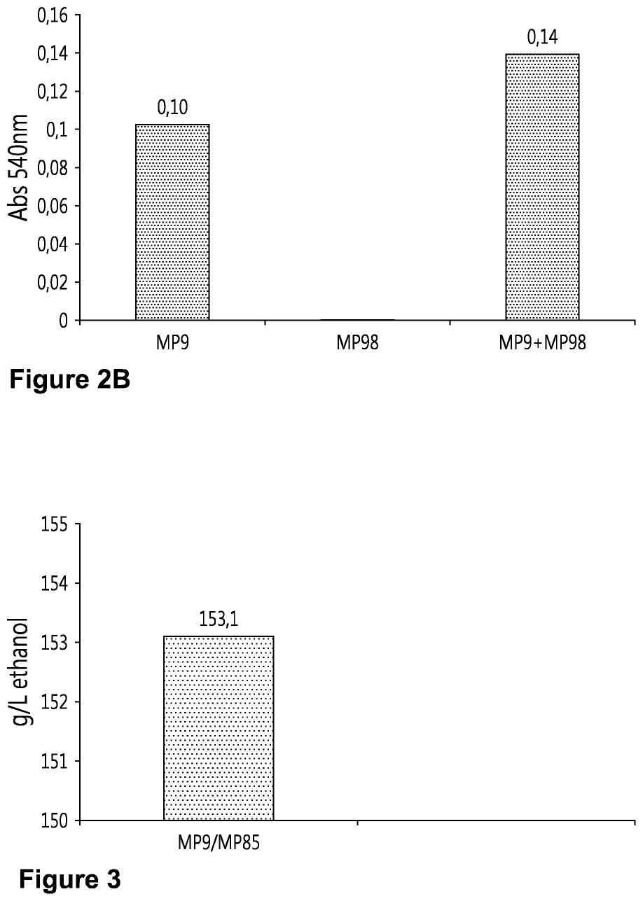

Then, the purified enzymes MP85 and MP98 were independently combined with a glucoamylase (MP9 encoded by the glu0111 gene from Saccharomycopsis fibuligera) and their ability to breakdown raw starch was determined, as indicated above. As shown on FIGS. 2A and B, a synergy in the amylase activity of MP9 and MP85 was observed.

To investigate performance in fermentation, purified yeast-made MP9 and MP85 were added in a weight ratio of 9:1 (MP9:MP85) in a fermentation medium inoculated with an industrial wild-type background strain. The fermentation medium comprised 32% total solids of corn flour and 500 ppm urea. The fermentation was conducted at a temperature between 30-32° C. for a period of 88 hrs. The results are shown in FIG. 3.

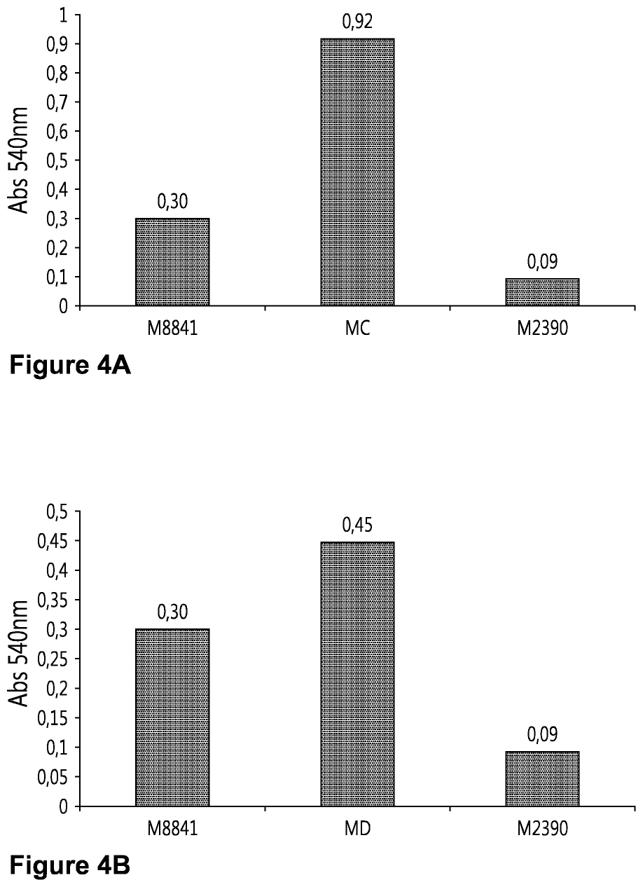

As a partially crystalline substrate, raw starch requires both glucoamylase and alpha-amylase activities for efficient and complete hydrolysis. MP85 and MP98 were each independently engineered into a S. cerevisiae strain genetically engineered to express the MP9 glucoamylase. The resulting strains (MC and MD) co-expressed gluco- and alpha-amylase genes and were characterized for the ability to hydrolyze raw corn starch, as indicated above. As shown on FIG. 4, the co-expression of a gluco- and an alpha-amylase resulted in a significant increase in secreted activity on raw corn starch.

While the invention has been described in connection with specific embodiments thereof, it will be understood that the scope of the claims should not be limited by the preferred embodiments set forth in the examples, but should be given the broadest interpretation consistent with the description as a whole.

1. A first polypeptide composition comprising a first polypeptide having alpha-amylase activity, for use in combination with a second polypeptide having glucoamylase activity on raw starch, wherein:
the first polypeptide is an alpha-amylase polypeptide, an alpha-amylase variant or an alpha-amylase fragment;
the alpha-amylase polypeptide comprises the amino acid sequence set forth in SEQ ID NO: 2;
the alpha-amylase variant has at least 70% amino acid sequence identity with the alpha-amylase polypeptide and has alpha-amylase activity;
the alpha-amylase fragment has at least 70% amino acid sequence identity with the alpha-amylase polypeptide and has alpha-amylase activity; and
the first polypeptide composition comprises either (i) the first polypeptide in a purified form, or (ii) the first polypeptide and a first recombinant host cell that expresses the first polypeptide, said first recombinant host cell comprising a first genetic modification allowing production of the alpha-amylase polypeptide, the alpha-amylase variant or the alpha-amylase fragment.

2. The first polypeptide composition of claim 1 which comprises the first polypeptide and the first recombinant host cell that expresses the first polypeptide.

3. The first polypeptide composition of claim 2, wherein the first recombinant host cell is a recombinant yeast host cell.

4. The first polypeptide composition of claim 3, wherein the first recombinant yeast host cell is a cell of genus Saccharomyces.

5. The first polypeptide composition of claim 4, wherein the first recombinant yeast host cell is Saccharomyces cerevisiae cell.

6. The first polypeptide composition of claim 1 which further comprises a second polypeptide composition comprising the second polypeptide having glucoamylase activity on raw starch, wherein the second polypeptide having glucoamylase activity is a glucoamylase polypeptide, a glucoamylase variant or a glucoamylase fragment and wherein:
the glucoamylase polypeptide comprises the amino acid sequence s forth in either SEQ ID NO: 5 or SEQ ID NO: 6;
the glucoamylase variant has at least 70% amino acid sequence identity with the glucoamylase polypeptide and has glucoamylase activity; and
the glucoamylase fragment has at least 70% amino acid sequence identity with the glucoamylase polypeptide and has glucoamylase activity.

7. The first polypeptide composition of claim 1, wherein the first recombinant host cell further comprises a second genetic modification selected from the group consisting of a genetic modification for reducing production of one or more native enzymes that function to produce glycerol or regulate glycerol synthesis, a genetic modification for allowing production of the second polypeptide having glucoamylase activity, and a genetic modification for reducing production of one or more native enzymes that function to catabolize formate.

8.-13. (canceled)

14. The first polypeptide composition of claim 1, wherein the alpha-amylase variant comprises the amino acid sequence set forth in SEQ ID NO: 1, SEQ ID NO: 2, SEQ ID NO: 7 or SEQ ID NO: 8.

15. The first polypeptide composition of claim 6 in which the second polypeptide composition comprises either (i) the second polypeptide having glucoamylase activity in a purified form, or (ii) the second polypeptide having glucoamylase activity and a second recombinant host cell that expresses the second polypeptide, said second recombinant host cell comprising a second genetic modification allowing production of the glucoamylase polypeptide, the glucoamylase variant or the glucoamylase fragment.

16.-25. (canceled)

26. A population of recombinant host cells, comprising:
(a) a first recombinant host cell that expresses a first polypeptide having alpha-amylase activity, for use in combination with a second polypeptide having glucoamylase activity on raw starch, wherein:
the first polypeptide is an alpha-amylase polypeptide, an alpha-amylase variant or an alpha-amylase fragment;
the alpha-amylase polypeptide comprises the amino acid sequence set forth in SEQ ID NO: 2;
the alpha-amylase variant has at least 70% amino acid sequence identity with the alpha-amylase polypeptide and has alpha-amylase activity;
the alpha-amylase fragment has at least 70% amino acid sequence identity with the alpha-amylase polypeptide and has alpha-amylase activity,
said first recombinant host cell comprising a first genetic modification allowing production of the alpha-amylase polypeptide, the alpha-amylase variant or the alpha-amylase fragment; and
(b) a second recombinant host cell that expresses a second polypeptide having glucoamylase activity on raw starch, wherein:
the second polypeptide is a glucoamylase polypeptide, a glucoamylase variant or a glucoamylase fragment;
the glucoamylase polypeptide comprises the amino acid sequence set forth in either SEQ ID NO: 5 or SEQ ID NO: 6;
the glucoamylase variant has at least 70% amino acid sequence identity with the glucoamylase polypeptide and has glucoamylase activity; and
the glucoamylase fragment has at least 70% amino acid sequence identity with the glucoamylase polypeptide and has glucoamylase activity:
said second recombinant host cell comprising a second genetic modification allowing production of the glucoamylase polypeptide, the glucoamylase variant or the glucoamylase fragment.

27. A method for hydrolyzing starch in a raw form to make a fermentation product, the method comprising fermenting a medium with a first polypeptide composition and a second polypeptide composition,
wherein the first polypeptide composition comprises a first polypeptide having alpha-amylase activity, for use in combination with a second polypeptide having glucoamylase activity on raw starch, wherein:
the first polypeptide is an alpha-amylase polypeptide, an alpha-amylase variant or an alpha-amylase fragment;
the alpha-amylase polypeptide comprises the amino acid sequence set forth in SEQ ID NO: 2;
the alpha-amylase variant has at least 70% amino acid sequence identity with the alpha-amylase polypeptide and has alpha-amylase activity;
the alpha-amylase fragment has at least 70% amino acid sequence identity with the alpha-amylase polypeptide and has alpha-amylase activity; and
the first polypeptide composition comprises either (i) the first polypeptide in a purified form, or (ii) the first polypeptide and a first recombinant host cell that expresses the first polypeptide, said first recombinant host cell comprising a first genetic modification allowing production of the alpha-amylase polypeptide, the alpha-amylase variant or the alpha-amylase fragment; and
wherein the second polypeptide composition comprises the second polypeptide having glucoamylase activity on raw starch, wherein:
the second polypeptide having glucoamylase activity is a glucoamylase polypeptide, a glucoamylase variant or a glucoamylase fragment and wherein:
the glucoamylase polypeptide comprises the amino acid sequence set forth in either SEQ ID NO: 5 or SEQ ID NO: 6:
the glucoamylase variant has at least 70% amino acid sequence identity with the glucoamylase polypeptide and has glucoamylase activity;
the glucoamylase fragment has at least 70% amino acid sequence identity with the glucoamylase polypeptide and has glucoamylase activity, and
the second polypeptide composition comprises either (i) the second polypeptide having glucoamylase activity in a purified form, or (ii) the second polypeptide having glucoamylase activity and a second recombinant host cell that expresses the second polypeptide, said second recombinant host cell comprising a second genetic modification allowing production of the glucoamylase polypeptide, the glucoamylase variant or the glucoamylase fragment.

28. The method of claim 27, wherein the fermentation product is ethanol.

29. The method of claim 27, wherein the medium comprises raw starch.

30. The medium of claim 27, wherein the medium is derived from corn.

31. A method for hydrolyzing starch in a raw form to make a fermentation product, the method comprising fermenting a medium with the population of recombinant host cells defined in claim 26.


SEQUENCE LISTING (8 sequences)

SEQ ID NO: 1 - protein, 652 aa, Artificial Sequence
  AMYE of Bacillus amyloliquefaciens with signal sequence from SUC2 gene of Saccharomyces cerevisiae
MLLQAFLFLLAGFAAKISAGPAAANAETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQVYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQVFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYTFVKKDPSQTNIIGYQNPDHWGQVNAYIYKHDGGRAIELTGSWPGKAMTKNANGMYTLTLPENTDTANAKVIFNNGSAQVPGQNQPGFDYVQNGLYNNSGLNGYLPH

SEQ ID NO: 2 - protein, 633 aa, Bacillus amyloliquefaciens
GPAAANAETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQVYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQVFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYTFVKKDPSQTNIIGYQNPDHWGQVNAYIYKHDGGRAIELTGSWPGKAMTKNANGMYTLTLPENTDTANAKVIFNNGSAQVPGQNQPGFDYVQNGLYNNSGLNGYLPH

SEQ ID NO: 3 - protein, 495 aa, Artificial Sequence
  ALP1 of Saccharomycopsis fibuligera with signal sequence of SUC2 gene from Saccharomyces cerevisiae
MLLQAFLFLLAGFAAKISAQPVTLFKRETNADKWRSQSIYQIVTDRFARTDGDTSASCNTEDRLYCGGSFQGIIKKLDYIKDMGFTAIWISPVVENIPDNTAYGYAYHGYWMKNIYKINENFGTADDLKSLAQELHDRDMLLMVDIVTNHYGSDGSGDSIDYSEYTPFNDQKYFHNYCLISNYDDQAQVQSCWEGDSSVALPDLRTEDSDVASVFNSWVKDFVGNYSIDGLRIDSAKHVDQGFFPDFVSASGVYSVGEVFQGDPAYTCPYQNYIPGVSNYPLYYPTTRFFKTTDSSSSELTQMISSVASSCSDPTLLTNFVENHDNERFASMTSDQSLISNAIAFVLLGDGIPVIYYGQEQGLSGKSDPNNREALWLSGYNKESDYYKLIAKANAARNAAVYQDSSYATSQLSVIFSNDHVIATKRGSVVSVFNNLGSSGSSDVTISNTGYSSGEDLVEVLTCSTVSGSSDLQVSIQGGQPQIFVPAKYASDICS

SEQ ID NO: 4 - protein, 476 aa, Saccharomyces barnetti
QPVTLFKRETNADKWRSQSIYQIVTDRFARTDGDTSASCNTEDRLYCGGSFQGIIKKLDYIKDMGFTAIWISPVVENIPDNTAYGYAYHGYWMKNIYKINENFGTADDLKSLAQELHDRDMLLMVDIVTNHYGSDGSGDSIDYSEYTPFNDQKYFHNYCLISNYDDQAQVQSCWEGDSSVALPDLRTEDSDVASVFNSWVKDFVGNYSIDGLRIDSAKHVDQGFFPDFVSASGVYSVGEVFQGDPAYTCPYQNYIPGVSNYPLYYPTTRFFKTTDSSSSELTQMISSVASSCSDPTLLTNFVENHDNERFASMTSDQSLISNAIAFVLLGDGIPVIYYGQEQGLSGKSDPNNREALWLSGYNKESDYYKLIAKANAARNAAVYQDSSYATSQLSVIFSNDHVIATKRGSVVSVFNNLGSSGSSDVTISNTGYSSGEDLVEVLTCSTVSGSSDLQVSIQGGQPQIFVPAKYASDICS

SEQ ID NO: 5 - protein, 515 aa, Saccharomycopsis fibuligera
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 6 - protein, 515 aa, Artificial Sequence
  A40N variant of SEQ ID NO 5
MIRLTVFLTAVFAAVASCVPVELDKRNTGHFQAYSGYTVNRSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 7 - protein, 652 aa, Artificial Sequence
  K34N variant of SEQ ID NO 1
MLLQAFLFLLAGFAAKISAGPAAANAETANKSNNVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQVYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQVFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYTFVKKDPSQTNIIGYQNPDHWGQVNAYIYKHDGGRAIELTGSWPGKAMTKNANGMYTLTLPENTDTANAKVIFNNGSAQVPGQNQPGFDYVQNGLYNNSGLNGYLPH

SEQ ID NO: 8 - protein, 633 aa, Artificial Sequence
  K34N variant of SEQ ID NO 2
GPAAANAETANKSNNVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQVYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQVFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYTFVKKDPSQTNIIGYQNPDHWGQVNAYIYKHDGGRAIELTGSWPGKAMTKNANGMYTLTLPENTDTANAKVIFNNGSAQVPGQNQPGFDYVQNGLYNNSGLNGYLPH